(19) United States
(12) Patent Application Publication
(10) Pub. No.: US 2022/0152095 A1
(43) Pub. Date: May 19, 2022

(54) ELECTROLYTE COMPOSITION AND METHODS OF USE

(71) Applicant: Nutreco IP Assets B.V., Boxmeer (NL)

(72) Inventors: Juliette Nina Roxanne WILMS, Boxmeer (NL); Javier MARTÍN-TERESO LÓPEZ, Boxmeer (NL)

(21) Appl. No.: 17/587,463

(22) Filed: Jan. 28, 2022

Related U.S. Application Data
(63) Continuation of application No. PCT/EP2020/071348, filed on Jul. 29, 2020.

(30) Foreign Application Priority Data
Jul. 29, 2019 (EP) 19188823.9

(51) Int. Cl. A61K 33/14; A23K 50/10; A23K 50/60; A23K 20/22; A23K 20/163; A23K 20/105; A61K 31/7004; A61K 47/12; A61P 3/12; A61K 9/00; A61K 9/08

(52) U.S. Cl. CPC A61K 33/14; A23K 50/10; A23K 50/60; A23K 20/22; A23K 20/163; A61K 9/08; A61K 31/7004; A61K 47/12; A61P 3/12; A61K 9/0053; A23K 20/105

(57) ABSTRACT

The present invention pertains to the field of oral electrolyte solutions (OES), particularly OES for calves. Particularly the present invention is in the fields of reducing diarrhea, reducing body weight loss, reducing fecal water loss and/or improving blood acid-base balance in a calf suffering from diarrhea.

### CROSS-REFERENCE TO RELATED APPLICATIONS

The present application is a Continuation of International Patent Application No. PCT/EP2020/071348, filed Jul. 29, 2020, which claims priority to Europe Patent Application No. 19188823.9 filed Jul. 29, 2019; the entire contents of all of which are hereby incorporated by reference.

### FIELD OF THE INVENTION

The present invention pertains to the field of oral electrolyte solutions (OES), particularly OES for calves. Particularly the present invention is in the fields of reducing diarrhea, reducing body weight loss, reducing fecal water loss and/or improving blood acid-base balance in a calf suffering from diarrhea.

### BACKGROUND OF THE INVENTION

Complications associated with neonatal diarrhea remain the most common cause of death in calves. Regardless of the cause of the diarrhea, diarrhea causes increased fecal water and electrolyte losses. This process results in dehydration, strong ion acidosis, electrolyte abnormalities, and often, a negative energy balance.

Although much research has been done in the field of oral rehydration therapy to mitigate the severity of dehydration and metabolic acidosis, which is a process that leads to a low pH in the blood as a result of dehydration, considerable variation exists in the composition and effectiveness of commercially available OES for calves.

Commercially available OES comprise sodium, potassium, and chloride ions. It is recommended that, for calves, the sodium concentration should be in the range of 90-130 mM. It is commonly recognized that products containing sodium at concentrations lower than 90 mM would not be able to adequately correct dehydration. It is recommended that OES should be hypertonic, having an osmolality as high as about 400-600 mOsm/kg, because hypertonic OES are thought to supply more energy to calves than products with a lower tonicity (Smith and Berchtold, 2014).

Such commercially available OES however, are associated with concerning health issues, such as hypernatremia, which is related to high sodium levels in the blood, with clinical signs such as digestive tract upsets, central nervous system dysfunction and death in acute cases. The excess of solutes present in hypertonic OES may also result in osmotic diarrhea and delayed abomasal emptying rates.

It is an object of the present invention to provide an improved OES for non-human animals, preferably young non-human animals, suffering from diarrhea.

### SUMMARY OF THE INVENTION

The present inventors have found that sodium ions concentration below the recommended range of 90-130 mM, chloride ion concentration range different from the recommended range of 40-80 mM, and an osmolality not higher than 350 mM, result in adequate rehydration and correction of metabolic acidosis of calves suffering from diarrhea, leading to reduction of diarrhea, body weight loss, fecal water loss and improvement of blood acid-base balance in a calf suffering from diarrhea.

In a first aspect, the present invention relates to a liquid electrolyte composition comprising: 60-88 mM sodium ions; 20-40 mM potassium ions; 20-50 mM, preferably 20-38 mM, chloride ions; 50-90 mM of an alkalinizing agent, which preferably comprises propionate and/or acetate; one or more sugars, preferably comprising lactose; said composition having an osmolality in the range of 200-350 mOsm/kg, preferably 215-330 mOsm/kg, more preferably 230-315 mOsm/kg, even more preferably 230-306 mOsm/kg, yet more preferably 240-280, and having a strong ion difference (SID) in the range of 60-80 mM.

In a further aspect, the present invention relates to a solid composition comprising sodium chloride; potassium chloride; alkalinizing agent, preferably comprising propionate and/or acetate, more preferably sodium propionate and/or sodium acetate; one or more sugars, preferably comprising lactose; said solid composition being intended for reconstitution in water, and which solid composition upon reconstitution in water provides the electrolyte solution as taught herein.

In an embodiment, the solid composition is in the form of a powder.

In a further aspect, the present invention relates to a method for rehydrating a non-human animal suffering from diarrhea, comprising administering to said non-human animal a composition as taught herein.

In another aspect, the present invention relates to a method for reducing diarrhea in a non-human animal comprising administering to a non-human animal suffering from diarrhea a composition as taught herein.

In another aspect, the present invention relates to a method for reducing body weight loss in a non-human animal suffering from diarrhea comprising administering to said non-human animal a composition as taught herein.

In another aspect, the present invention relates to a method for reducing fecal water loss in a non-human animal suffering from diarrhea comprising administering to said non-human animal a composition as taught herein.

In another aspect, the present invention relates to a method for improving blood acid-base balance in a non-human animal suffering from diarrhea comprising administering to said non-human animal a composition as taught herein.

### DETAILED DESCRIPTION OF THE INVENTION

### General Definitions

In the following description and examples, a number of terms are used. In order to provide a clear and consistent understanding of the specification and claims, including the scope to be given to such terms, the following definitions are provided. Unless otherwise defined herein, all technical and scientific terms used have the same meaning as commonly understood by one of ordinary skill in the art to which this invention belongs. The disclosures of all publications, patent applications, patents and other references cited herein are incorporated herein in their entirety by reference.

The terms ‘electrolyte composition’, ‘oral electrolyte solution’, ‘electrolyte solution’, ‘oral electrolyte solution’ or ‘OES’, as used herein, refer to oral rehydration solutions which can be used to manage fluid loss due to diarrhea. Generally, these compositions consist of salts, water and sugar, and help to replenish fluids and electrolytes which have been lost from the body. They help the body to absorb water and electrolytes to prevent further dehydration.

The term ‘alkalinizing agents’, as used herein, refers to compounds that are used to manage situations associated with low blood pH. For example, they can be used when an animal suffers from metabolic acidosis, to increase blood pH. Administration of an alkalinizing agent results in a lowering of plasma and extracellular [H⁻], with a concurrent increase in concentration of alkali ions, such as for example [HCO₃⁻]. Examples of alkalinizing agents are sodium propionate, sodium bicarbonate, potassium citrate, calcium carbonate, sodium lactate, sodium acetate or calcium acetate.

The term ‘osmolality’, as used herein, refers to the number of osmoles (Osm) of solute per kilogram of solvent (Osm/kg=1000 mOsm/kg). In an oral electrolyte solution for animals for example, the osmolality is the number of osmoles of solute per kilogram of water. Also body fluids, like blood or milk, have a certain osmolality value.

The term ‘tonicity’, as used herein, refers to the effective osmolality and is equal to the sum of the concentrations of the solutes which have the capacity to exert an osmotic force across a membrane. Tonicity is a property of a solution in reference to a particular membrane. It is also defined as the ability of an extracellular solution to make water move into or out of a cell by osmosis. If the extracellular fluid has less solutes that cannot cross the membrane than inside the cell, than the fluid is said to be hypotonic, which means, net flow of water will be into the cell. If the extracellular fluid has more solutes that cannot cross the membrane than inside the cell, than the fluid is said to be hypertonic, which means, net flow of water will be out of the cell. With an isotonic extracellular fluid the amounts of solutes that cannot cross the membrane will be the same in the cell compared to the extracellular fluid. In that case there will be no net movement of water into or out of the cell. For tonicity, body fluids (such as milk or blood) are taken as a reference, typically having an osmolality value of around 300 mOsm per kg fluid. In practice the skilled person knows that that value can be slightly higher or lower, such as 300 plus or minus 1, 2, 3, 4, 5, 6, 7, 8, 9 or 10 mOsm/kg. Fluids with higher tonicity are called hypertonic; fluids with lower tonicity are called hypotonic.

The term ‘strong ion difference’ or ‘SID’, as used herein, refers to the difference between the positively- and negatively-charged strong ions in plasma or any other liquids. Strong ions are cations and anions that exist as charged particles dissociated from their partner ions at physiologic pH. These ions are “strong” because their ionization state is independent of pH. When SID of an oral electrolyte solution is high it may alkalinize the blood of an animal. When SID is negative it acidifies the blood of the animal. In a liquid such as a drinking water the SID may be calculated as SID=[Na⁺]+[K⁺]-[Cl⁻] and is expressed in mEq/L.

The term ‘blood base excess’ or ‘blood BE’, as used herein, refers to the most commonly accepted parameter to evaluate the acid-base status of the blood. Blood pH is tightly regulated by the animal and therefore difficult to use for investigating the amplitude of acid-base changes. Blood base excess is defined as the amount of strong acid, e.g. HCl or H₂SO₄, that must be added to each liter of fully oxygenated blood to return the pH to 7.40 at a temperature of 37° C. and a p002 of 40 mmHg (5.3 kPa). The formula for base deficit is as follows: base excess=0.02786×p002×10^{(pH-6.1)}+13.77×pH-124.58. In practice, blood BE can easily be measured by a blood analyzer instrument, like for example an i-Stat system. Good blood BE values for calves are between about 2.6 to about 10.8 mmol/L (Dillane et al., 2018). A negative BE value indicates metabolic acidosis, however, the skilled person knows that some variation is possible in practice, and the value may also be just above zero, such as 1, 1.5 or 2 and the like.

The term ‘blood acid-base balance’, also known as ‘blood acid-base status’, as used herein, refers to the balance between acids and bases in the blood. For the health of animals it is important that a disturbed blood acid-base balance, as a result of, for example, diarrhea, needs to be restored. Blood acid-base balance parameters (pH, blood BE, and HCO3) were determined in whole blood, using a blood gas analyzer (VetScan I-STAT®1, ref: 600-7015). One drop of blood from a lithium heparin tube was inserted in a cartridge and analysed immediately using a blood gas analyser.

The term ‘diarrhea’, as used herein, refers to a situation of an increase in the frequency of bowel movements, an increase in the looseness of stool, or both. It is caused by increased secretion of fluid into the intestine, reduced absorption of fluid from the intestine or rapid passage of stool through the intestine. Calf diarrhea and complications associated with calf diarrhea are the leading cause of calf mortality worldwide. Complications of diarrhea include, among others, dehydration and electrolyte- or mineral abnormalities.

The terms ‘to improve’ or ‘improving’ as used herein refer to the ability to bring into a more desirable state or condition. Someone or something might, for example, become better or might get better properties or quality. The ability to make things better is also covered in a sense of the ability to ameliorate, like improving a bad situation or quality, or repairing bad or not working properties.

The terms ‘to increase’ and ‘increased level’ and the terms ‘to decrease’ and ‘decreased level’ refer to the ability to increase or decrease a particular amount or number. A level in a test sample may be increased or decreased when it is at least 5%, such as 10%, 15%, 20%, 25%, 30%, 35%, 40%, 45%, 50% higher or lower, respectively, than the corresponding level in a control sample or reference sample. Similar to the term ‘decrease’ is the term ‘reduce’. A reduction also means a decrease in for example an amount, a degree, or size. In a context of for example ‘reducing diarrhea’, also terms may be used similar to ‘reducing’, such as ‘mitigating’ or ‘alleviating’, meaning that something bad becomes less severe or serious.

The term ‘about’, as used herein indicates a range of normal tolerance in the art, for example within 2 standard deviations of the mean. The term “about” can be understood as encompassing values that deviate at most 10%, 9%, 8%, 7%, 6%, 5%, 4%, 3%, 2%, 1%, 0.5%, 0.1%, 0.05%, or 0.01% of the indicated value.

The terms “comprising” or “to comprise” and their conjugations, as used herein, refer to a situation wherein said terms are used in their non-limiting sense to mean that items following the word are included, but items not specifically mentioned are not excluded. It also encompasses the more limiting verb “to consist essentially of” and “to consist of”.

Reference to an element by the indefinite article “a” or “an” does not exclude the possibility that more than one of the elements is present, unless the context clearly requires that there be one and only one of the elements. The indefinite article “a” or “an” thus usually means “at least one”.

### Compositions

In a first aspect, the present invention relates to a liquid electrolyte composition comprising: 60-88 mM, preferably 63-87 mM, more preferably 66-86 mM, even more preferably 69-85 mM sodium ions; 20-40 mM, preferably 20-38 mM, more preferably 22-35 mM, even more preferably 24-32 mM potassium ions; 20-50 mM, preferably 23-44 mM, more preferably 26-38 mM, even more preferably 29-36 mM chloride ions; 50-90 mM, preferably 55-85 mM, more preferably 60-80 mM, even more preferably 65-75 mM of an alkalinizing agent, which preferably comprises propionate and/or acetate; one or more sugars, preferably comprising lactose; said composition having an osmolality in the range of 200-350 mOsm/kg, preferably 215-330 mOsm/kg, more preferably 230-315 mOsm/kg, more preferably 230-306 mOsm/kg, more preferably 230-300 mOsm/kg, more preferably 235-295 mOsm/kg, more preferably 235-290 mOsm/kg, more preferably 240-285 mOsm/kg, yet more preferably 240-280 mOsm/kg, and having a strong ion difference (SID) in the range of 60-80 mM, preferably 65-80 mM, more preferably 70-80 mM.

In an embodiment, the alkalinizing agent is selected from propionate, acetate, carbonate, bicarbonate, citrate and/or lactate. In an embodiment, the alkalinizing agent is selected from propionate and/or acetate.

In an embodiment, the sugar is selected from a monosaccharide and/or a disaccharide. In an embodiment, the sugar is selected from dextrose, fructose, glucose, galactose and/or lactose. In an embodiment, the sugar is selected from lactose and/or dextrose. In an embodiment, the glucose-to-sodium ratio is in a range of 0.2-5, preferably in a range of 0.4-4, more preferably in a range of 0.7-2.5, even more preferably in a range of 0.9-1.3; wherein, in case of a disaccharide (e.g. lactose), one unit of a disaccharide counts for two units of glucose.

Sodium ions, potassium ions, chloride ions, alkalinizing agent and/or one or more sugars may be added in order to get the composition as taught herein either (i) by using one or more solutions comprising one or more of sodium-, potassium- and/or chloride ions, alkalinizing agent and/or one or more sugars; or (ii) by dissolving one or more of a sodium-, potassium- and/or chloride salt, alkalinizing agent and/or one or more sugars, in a fluid, preferably an aqueous fluid; or by a combination of i and ii.

In a further aspect, the present invention relates to a solid composition comprising sodium chloride; potassium chloride; alkalinizing agent, preferably comprising propionate and/or acetate, more preferably sodium propionate and/or sodium acetate; one or more sugars, preferably comprising lactose; said solid composition being intended for reconstitution in water, which upon reconstitution in water provides the electrolyte solution as taught herein. In an embodiment, the solid composition is provided together with instructions for reconstitution into water.

In an embodiment, the alkalinizing agent is selected from propionate, acetate, carbonate, bicarbonate, citrate and/or lactate. In an embodiment, the alkalinizing agent is selected from sodium propionate, sodium bicarbonate, potassium citrate, calcium carbonate, sodium lactate, sodium acetate and/or calcium acetate. In an embodiment, the alkalinizing agent is selected from propionate and/or acetate. In an embodiment, the alkalinizing agent is selected from sodium propionate and/or sodium acetate.

In an embodiment, the sugar is selected from a monosaccharide and/or a disaccharide. In an embodiment, the sugar is selected from dextrose, fructose, glucose, galactose and/or lactose. In an embodiment, the sugar is selected from lactose and/or dextrose. In an embodiment, the glucose-to-sodium ratio is in a range of 0.2-5, preferably in a range of 0.4-4, more preferably in a range of 0.7-2.5, even more preferably in a range of 0.9-1.3; wherein, in case of a disaccharide (e.g. lactose), one unit of a disaccharide counts for two units of glucose.

In an embodiment, the solid composition is in the form of a powder.

The solid composition is intended for reconstitution in water and may for example be contained in a can, box, bottle or bucket or the like, from which the composition may be added to a fluid using a scoop or spoon, such as a dosage scoop or spoon, or the like, in order to end up with the electrolyte solution of the invention. The solid composition may be contained in a single-use packaging, such as a sachet, intended for reconstitution in a certain volume of fluid, such as water. In an embodiment the sachet comprises an amount of solid composition intended for reconstitution in about 2 liters of water.

### Methods and Uses of the Composition

In an aspect, the present invention relates to a method for rehydrating a non-human animal suffering from diarrhea, comprising administering to said non-human animal a composition as taught herein.

In a further aspect, the present invention relates to a method for reducing diarrhea in a non-human animal comprising administering to a non-human animal suffering from diarrhea a composition as taught herein.

In another aspect, the present invention relates to a method for reducing body weight loss in a non-human animal suffering from diarrhea comprising administering to said non-human animal a composition as taught herein.

In another aspect, the present invention relates to a method for reducing fecal water loss in a non-human animal suffering from diarrhea comprising administering to said non-human animal a composition as taught herein.

In another aspect, the present invention relates to a method for improving blood acid-base balance in a non-human animal suffering from diarrhea comprising administering to said non-human animal a composition as taught herein. It was found by the inventors that present invention has a stronger alkalinizing capacity compared to products known in the market, which led to a surprising improvement of the blood acid-base balance. For the health of animals it is important that a disturbed blood acid-base balance, as a result of for example diarrhea, needs to be restored.

The non-human animal is preferably a young non-human animal, more preferably a piglet, foal or a calf. Most preferably the non-human animal is a calf.

In an embodiment, the electrolyte solution is prepared by addition of the solid composition as taught herein to water. In an embodiment, the electrolyte solution is prepared by reconstituting an amount of the solid composition as taught herein, in about 2 liters of water.

In an embodiment, the liquid electrolyte composition taught herein is provided to a calf on a twice-daily basis. Preferably, about 1,5-2,5 liters of the liquid electrolyte composition taught herein is offered to a calf on a twice-daily basis in between milk meals, such as calf milk replacer.

The present invention is further illustrated, but not limited, by the following example.

From the above discussion and the example, one skilled in the art can ascertain the essential characteristics of the present invention, and without departing from the teaching and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### EXAMPLE

### Example 1

A total of 72 Holstein-Friesian diarrheic calves (22±7 days of age) with naturally occurring diarrhea were selected at the location of origin based on the severity of metabolic acidosis assessed by blood base excess (BE). Calves were allocated to blocks of four animals based on blood BE one day after arrival, which means that animals with similar blood BE were grouped together. Within a block, calves were randomly assigned to one of four treatments (see also Table 1 for compositions): 1) hypertonic OES with high Na and high dextrose (HYPER); 2) isotonic OES with low Na and low dextrose (ISO); 3) hypotonic OES with low Na and low lactose (HYPO) and 4) control of warm water containing 5 grams whey powder per liter as a placebo treatment, in order to get the same visual presentation as to the other treatments (CON). Each OES was formulated to have the same alkalinizing capacity (strong ion difference of 76 mEq/L, and propionate concentration of 72 mmol/L) and a glucose-to-sodium ratio of 1.1, wherein, in case of lactose, one unit of lactose counts for two units of glucose.

Treatments were administered twice daily over a 3-day period, in which calves were offered 2 L of treatment at 13:00 and 21:00 h. Calves were fed 2.5 L of milk replacer (MR) at 06:30 and 17:00 h, and had ad libitum access to water, except during treatment administration. All intakes were recorded daily. Blood samples were taken once daily at 11:00 h for a duration of 5 days. Feces and urine were quantitatively collected for a duration of 3 d and calves were weighed on d 1 and 5.

The following tables show the compositions used in the different treatments (table 1), body weight of the calves before and after treatments (table 2) and effects of treatments on water balance parameters (table 3) and on blood acid-base balance (table 4) in milk fed calves with naturally occurring diarrhea.

TABLE 1
Components of oral electrolyte solutions in the treatments.

| | Treatments² | | | |
|---|---|---|---|---|
| Item¹ | CON | ISO | HYPO | HYPER |
| Sugars | | | | |
| Lactose | 10 | 0 | 45 | 0 |
| Dextrose | 0 | 90 | 0 | 151 |
| Minerals | | | | |
| Sodium | 2 | 82 | 79 | 134 |
| Potassium | 3 | 27 | 27 | 27 |
| Chloride | 2 | 33 | 33 | 86 |
| Alkalinizing agent | | | | |
| Propionate | 0 | 72 | 72 | 72 |
| SID, mEq/L³ | 2 | 76 | 76 | 76 |
| Osmolality, mOsm/kg⁴ | 18 | 305 | 249 | 471 |

¹Expressed in mmol/L.
²HYPER = high sodium, high dextrose, ISO = low sodium, low dextrose, HYPO = low sodium, low lactose, CON = control.
³Effective strong ion difference = [Na⁺] + [K⁺] − [Cl⁻]
⁴Osmolality (in moles per kg of H₂O) was calculated according to Constable et al. (2009) by adding osmolality of carbohydrates (lactose, dextrose and galactose) and minerals (Na, K, Cl, P, Ca and Mg).

TABLE 2
Body weight of calves before and after treatment initiation (n = 72).

| | Treatment¹ | | | |
|---|---|---|---|---|
| | CON | ISO | HYPO | HYPER |
| Before treatment initiation | | | | |
| Age at day 1, days | 20.15 | 20.75 | 19.93 | 19.85 |
| Initial body weight (d1), kg | 43.63 | 44.89 | 45.07 | 44.62 |
| After treatment initiation | | | | |
| End Body weight (d5), kg | 44.55 | 45.70 | 46.23 | 44.80 |

¹Treatments included one control solution (CON, n = 18) and three oral electrolyte solutions: high sodium, high dextrose (HYPER, n = 18), low sodium, low dextrose (ISO, n = 18) and low sodium, low lactose (HYPO, n = 18).

Body weights of ISO and HYPO calves were higher than CON and HYPER calves on day 5 which may be an indication for improved rehydration of ISO and HYPO calves.

TABLE 3
The effect of oral administration of different electrolyte
solutions on water balance parameters intake, urinary losses
and fecal losses on day 1 of total collection in milk fed
calves with naturally occurring diarrhea (n = 72).

| Water balance parameters | Treatments¹ | | | |
|---|---|---|---|---|
| (g/kg BW/day) | CON | ISO | HYPO | HYPER |
| Intake | 139.05 | 179.14 | 180.71 | 164.21 |
| Urinary losses | 45.02 | 66.82 | 73.31 | 48.17 |
| Fecal losses | 43.90 | 30.97 | 22.68 | 37.14 |

¹Treatments included one control solution (CON, n = 18) and three oral electrolyte solutions: high sodium, high dextrose (HYPER, n = 18), low sodium, low dextrose (ISO, n = 18) and low sodium, low lactose (HYPO, n = 18).

Total water fluid intake on day 1 was higher for ISO and HYPO calves compared to CON or HYPER calves. Urinary water losses were higher in ISO and HYPO calves compared to CON and HYPER calves, whereas fecal water losses were lower for HYPO and ISO calves compared to CON and HYPER calves. As the purpose of the electrolyte solution taught herein is to reduce diarrhea, these results were in accordance with expectations: less water was excreted through the feces, whereas more water was excreted through urine.

TABLE 4
The effect of oral administration of different electrolyte
solutions on blood acid-base balance in milk
fed calves with naturally occurring diarrhea (n = 72).

| | Treatments² | | | |
|---|---|---|---|---|
| Item¹ | CON | ISO | HYPO | HYPER |
| Blood acid-base balance | | | | |
| pH | 7.33 | 7.41 | 7.42 | 7.35 |
| BE | −1.35 | 6.48 | 9.15 | 1.71 |
| HCO₃₋ | 24.58 | 31.20 | 33.51 | 27.17 |

¹Expressed in mmol/L unless specified otherwise.
²Treatments included one control solution (CON, n = 18) and three oral electrolyte solutions: high sodium, high dextrose (HYPER, n = 18), low sodium, low dextrose (ISO, n = 18) and low sodium, low lactose (HYPO, n = 18).

For the health of animals it is important that a disturbed blood acid-base balance related to metabolic acidosis, as a result of, for example, diarrhea, needs to be restored. That means that blood pH needs to be increased towards normal pH (like around 7.40); blood BE needs to be increased (between about 2.6 and 10.8 mmol/L); and HCO₃⁻ needs to be increased compared to control values as a measure for the blood becoming less acidic.

Blood acid-base balance (pH, BE, and HCO₃⁻) was determined in whole blood using a blood gas analyzer (VetScan I-STAT®, ref: 600-7015). Concentrations for blood acid-base balance are presented in Table 4. Blood pH, blood BE, and blood HCO3 were lower for CON and HYPER calves compared to ISO and HYPO calves.

Low tonicity OES are therefore more effective at maintaining and restoring blood acid-base balance than hypertonic OES or control treatment.

1. A liquid electrolyte composition having an osmolality in the range of 200-315 mOsm/kg, and having a strong ion difference (SID) in the range of 60 to 80 mM, and comprising:
(a) 60-88 mM sodium ions;
(b) 20-40 mM potassium ions;
(c) 20-50 mM chloride ions;
(d) 50-90 mM of an alkalinizing agent;
(e) one or more sugars;
said composition.

2. The liquid electrolyte composition according to claim 1, comprising 20-38 mM chloride ions.

3. The liquid electrolyte composition according to claim 1, wherein the alkalinizing agent comprises propionate and/or acetate.

4. The liquid electrolyte composition according to claim 1, wherein the sugars comprise lactose.

5. A solid composition, comprising:
(a) sodium chloride;
(b) potassium chloride;
(c) alkalinizing agent;
(d) one or more sugars;
wherein the solid composition, upon reconstitution in water, provides an electrolyte solution according to claim 1.

6. The solid composition according to claim 5, wherein the alkalinizing agent comprises propionate and/or acetate.

7. The solid composition according to claim 5, wherein the alkalinizing agent comprises sodium propionate and/or sodium acetate.

8. The solid composition according to claim 5, wherein the sugars comprise lactose.

9. The solid composition according to claim 5, in the form of a powder.

10. A method for rehydrating a non-human animal suffering from diarrhea, comprising administering to the non-human animal a composition according to claim 1.

11. A method for reducing diarrhea in a non-human animal, comprising administering to a non-human animal suffering from diarrhea a composition according to claim 1.

12. A method for reducing body weight loss in a non-human animal suffering from diarrhea, comprising administering to a non-human animal a composition according to claim 1.

13. A method for reducing fecal water loss in a non-human animal suffering from diarrhea, comprising administering to the non-human animal a composition according to claim 1.

14. A method for improving blood acid-base balance in a non-human animal suffering from diarrhea, comprising administering to the non-human animal a composition according to claim 1.
